SEQUENCE LISTING (2 sequences)

SEQ ID NO: 1 - protein, 11 aa, Artificial sequence
  Peptide
GRRAAPGRAGG

SEQ ID NO: 2 - protein, 11 aa, Artificial sequence
  Peptide
GRRAAPGRAGG

(19) United States
(12) Patent Application Publication
(10) Pub. No.: US 2016/0015771 A1
(43) Pub. Date: Jan. 21, 2016

(54) METHOD OF TREATING OPTIC NERVE DAMAGE, OPHTHALMIC ISCHEMIA OR OPHTHALMIC REPERFUSION INJURY

(71) Applicant: Curonz Holdings Company Limited, Auckland (NZ)

(72) Inventor: Frank SIEG, Wellsford (NZ)

(73) Assignee: Curonz Holdings Company Limited, Auckland (NZ)

(21) Appl. No.: 14/416,667

(22) PCT Filed: Jul. 26, 2013
(86) PCT No.: PCT/NZ2013/000132

§ 371 Date: Jan. 23, 2015

(30) Foreign Application Priority Data
Jul. 27, 2012 (NZ) 601512

(51) Int. Cl. A61K 38/08; A61K 9/00

(52) U.S. Cl. CPC A61K 38/08; A61K 9/0048

(57) ABSTRACT

The invention relates to a method of treating optic nerve damage, ophthalmic ischemia or ophthalmic reperfusion injury including the step of administering an effective amount of a peptide comprising the sequence: GlyArgArgAlaAlaProGlyArgAibGlyGly (SEQ ID NO:1) or the sequence GlyArgArgAlaAlaProGlyArgAibGlyGly-HN2 (SEQ ID NO:2) to a subject in need thereof.

### FIELD OF INVENTION

The invention relates to a method of treating optic nerve damage, ophthalmic ischemia or ophthalmic reperfusion injury.

### BACKGROUND ART

Progressive loss of retinal ganglion cells (RGCs) is a hallmark of traumatic or glaucoma-like injury of the optic nerve (Soto et al., 2008). Apart from the initial primary injury to retinal neurons caused by the neurodegenerative disease process there is a secondary apoptotic process assumed that is mediated by the elevation of excitotoxins like extracellular glutamate causing further damage to the retina (Prokosch et al., 2010). There is a strong need to identify neuroprotective substances that will be therapeutically effective in a clinical relevant setting. Brimonidine, an alpha-2A-adrenergic receptor agonist has been shown to be neuroprotective in formulations either topically or intraperitoneal (IP) applied within various ischemia-related optic nerve injury animal models (Weber et al., 2007; Yoles et al., 1999; Levkovitch-Verbin et al., 2000 and Loengren et al., 2006). It is known that Brimonidine is only active when administered in prophylactic fashion within in vivo rodent models of optic nerve ischemia but loses its effect dramatically in regard to promotion of retinal ganglion cell (RGC) survival even when applied minutes after ex vivo performed injuries of retinal tissue (Prokosch et al., 2010). So far, Brimodine has shown promising neuroprotective activities in rodent models of optic nerve damage but failed to do so when used in comparable human ophthalmic diseases.

It is an object of this invention to provide a method for use in such treatments comprising a neural regeneration peptide or to at least to provide a useful choice.

### STATEMENT OF INVENTION

In a first aspect the invention provides a method of treating or preventing optic nerve damage in a subject comprising administering an effective amount of a peptide comprising the sequence:

GlyArgArgAlaAlaProGlyArgAibGlyGly (SEQ ID NO:1) to an eye of a subject in need thereof.

In one embodiment the peptide consists of the 11 amino acid residue sequence:

GlyArgArgAlaAlaProGlyArgAibGlyGly

and ophthalmologically acceptable derivatives thereof. One such ophthalmically acceptable derivative includes the sequence wherein the C-terminus of the peptide is amidated to give:

(SEQ ID NO: 2)
GlyArgArgAlaAlaProGlyArgAibGlyGly-NH₂.

In one embodiment the administration step to the subject is by way of one or more topically applied eye drops. In another embodiment the administration step to the subject's eye may be by way of administration of a cream pr an ointment. In another embodiment the administration step to the subject's eye is by way of a liquid drop preparation applied to the conjunctival sac of the eye of the subject. In another embodiment the administration step to the subject's eye is by way of an intravitreal injection.

In one embodiment the method includes the step of administering an effective amount of a peptide comprising the sequence:

(SEQ ID NO: 1)
GlyArgArgAlaAlaProGlyArgAibGlyGly
or
(SEQ ID NO: 2)
GlyArgArgAlaAlaProGlyArgAibGlyGly-NH₂

to an eye of the subject in need thereof on an at least once a day basis.

In one embodiment the method includes the step of administering an effective amount of a peptide comprising the sequence:

(SEQ ID NO: 1)
GlyArgArgAlaAlaProGlyArgAibGlyGly

to an eye of the subject in need thereof on an at least once a day basis.

In one embodiment the methods defined above include the step of administering the peptide on an at least twice a day basis.

In one embodiment the subject is selected from the group consisting of: humans and companion animals.

In a second aspect the invention provides a method of treating or preventing an ophthalmic reperfusion injury in a subject comprising administering an effective amount of a peptide comprising the sequence:

GlyArgArgAlaAlaProGlyArgAibGlyGly (SEQ ID NO:1) to an eye of a subject in need thereof.

In one embodiment the peptide consists of the 11 amino acid residue sequence:

GlyArgArgAlaAlaProGlyArgAibGlyGly

and ophthalmologically acceptable derivatives thereof. One such ophthalmically acceptable derivative includes the sequence wherein the C-terminus of the peptide is amidated to give:

(SEQ ID NO: 2)
GlyArgArgAlaAlaProGlyArgAibGlyGly-NH₂.

In one embodiment the administration step to the subject is by way of one or more topically applied eye drops. In another embodiment the administration step to the subject's eye may be by way of administration of a cream or an ointment. In another embodiment the administration step to the subject's eye is by way of a liquid drop preparation applied to the conjunctival sac of the eye of the subject. In another embodiment the administration step to the subject's eye is by way of an intravitreal injection.

In one embodiment the method includes the step of administering an effective amount of a peptide comprising the sequence:

(SEQ ID NO: 1)
GlyArgArgAlaAlaProGlyArgAibGlyGly
or
(SEQ ID NO: 2)
GlyArgArgAlaAlaProGlyArgAibGlyGly-NH₂

to an eye of the subject in need thereof on an at least once a day basis.

In one embodiment the method includes the step of administering an effective amount of a peptide comprising the sequence:

(SEQ ID NO: 1)
GlyArgArgAlaAlaProGlyArgAibGlyGly

to an eye of a subject in need thereof on an at least once a day basis.

In one embodiment the methods defined above include the step of administering the peptide on an at least twice a day basis.

In one embodiment the subject is selected from the group consisting of: humans and companion animals.

In a third aspect the invention provides a method of treating or preventing ophthalmic ischemia in a subject comprising administering an effective amount of a peptide comprising the sequence:

GlyArgArgAlaAlaProGlyArgAibGlyGly (SEQ ID NO:1) to an eye of a subject in need thereof.

In one embodiment the peptide consists of the 11 amino acid residue sequence:

GlyArgArgAlaAlaProGlyArgAibGlyGly

and ophthalmologically acceptable derivatives thereof. One such ophthalmically acceptable derivative includes the sequence wherein the C-terminus of the peptide is amidated to give:

(SEQ ID NO: 2)
GlyArgArgAlaAlaProGlyArgAibGlyGly-NH₂.

In one embodiment the administration step to the subject is by way of one or more topically applied eye drops. In another embodiment the administration step to the subject's eye may be by way of administration of a cream or an ointment. In another embodiment the administration step to the subject's eye is by way of a liquid drop preparation applied to the conjunctival sac of the eye of the subject. In another embodiment the administration step to the subject's eye is by way of an intravitreal injection.

In one embodiment the method includes the step of administering an effective amount of a peptide comprising the sequence:

(SEQ ID NO: 1)
GlyArgArgAlaAlaProGlyArgAibGlyGly
or
(SEQ ID NO: 2)
GlyArgArgAlaAlaProGlyArgAibGlyGly-NH₂

to an eye of the subject in need thereof on an at least once a day basis.

In one embodiment the method includes the step of administering an effective amount of a peptide comprising the sequence:

(SEQ ID NO: 1)
GlyArgArgAlaAlaProGlyArgAibGlyGly

to an eye of the subject in need thereof on an at least once a day basis.

In one embodiment the method defined above includes the step of administering the peptide on an at least twice a day basis.

In one embodiment the subject is selected from the group consisting of: humans and companion animals.

In a fourth aspect the invention provides the use in the manufacture of a medicament of an effective amount of at least one of the peptides selected from:

(SEQ ID NO: 1)
GlyArgArgAlaAlaProGlyArgAibGlyGly
or
(SEQ ID NO: 2)
GlyArgArgAlaAlaProGlyArgAibGlyGly-NH₂

for treating or preventing
(i) optic nerve damage;
(ii) an ophthalmic reperfusion injury; or
(iii) ophthalmic ischemia in an eye of a subject in need thereof.

In one embodiment the medicament is adapted for topical ophthalmic administration.

In one embodiment the medicament is adapted for topical ophthalmic administration in the form of a cream or an ointment.

In one embodiment the medicament is adapted as a liquid formulation for application to the conjunctival sac of the eye of the subject.

In another embodiment medicament is adapted as a liquid formulation for application to the subject's eye by way of an intravitreal injection.

In one embodiment the medicament is adapted for at least once daily administration.

In one embodiment the medicament is adapted for at least twice daily administration.

In one embodiment the subject is selected from the group consisting of: humans and companion animals.

It will be understood from the following description that the effective amount of the peptide of SEQ ID NO:1 or SEQ ID NO: 2 is indicated to be in the range of 2-20 nanogram dose amounts when administered in rodent models of optic nerve injury and in the range of 20-200 nanogram dose amounts when administered to larger subjects such as dogs or humans.

In the description and claims of this specification the following acronyms, terms and phrases have the meaning provided:

“Effective amount” means an amount effective to treat or prevent optic nerve damage; an ophthalmic reperfusion injury; or ophthalmic ischemia in a given subject.

“Functionally similar amino acid” means an amino acid with similar properties according to the following groupings:
Neutral-weakly hydrophobic (Ala, Gly, Pro, Ser, Thr)
Hydrophilic-Acid Amine (Asn, Asp, Gln, Glu)
Hydrophilic-Basic (Arg, His, Lys)
Hydrophobic (Ile, Met, Leu, Val)
Hydrophobic-Aromatic (Phe, Trp, Tyr)
Cross-linking (Cys)

“Ophthalmologically acceptable derivatives” means derivatives of the peptide defined in SEQ ID NO:1 obtained by amidation, acylation, alkylation, carboxylation, glycosylation, phosphorylation, prenylation, salification, sulfation, or a combination thereof, that are suitable for inclusion in a composition for administration to the eye.

“Ophthalmologically acceptable excipients” means excipients selected from stabilizing agents, surfactants, buffering agents, chelating agents, viscosity agents, tonicity agents and preservative agents that are suitable for inclusion in a composition for administration to the eye.

In the description and claims of this specification the nucleotides and amino acids of biosequences (nucleic acids and peptides) are identified in accordance with Tables 1 to 4 of Annex C, Appendix 2 of the PCT Administrative Instructions (as in force from Jan. 1, 2010).

The invention will now be described with reference to embodiments or examples and the figures of the accompanying drawings pages.

### BRIEF DESCRIPTION OF DRAWINGS

FIG. 1. Shows a plot of a-wave amplitude results wherein SEQ ID NO: 1 also known as NRP2945 was administered as an eye drop (twice daily) in one group of rats starting at 30 min after optic nerve ligation/reperfusion injury compared to prophylatically administered Brimonidine to another group of rats.

FIG. 2. Shows a plot of b-wave results where SEQ ID NO: 1, also known as NRP2945, was administered as an eye drop (twice daily) in one group of rats starting at 30 min after optic nerve ligation/reperfusion injury compared to the prophylactically administered Brimonidine to another group of rats.

FIG. 3. Shows a plot of retinal ganglion cells (RCG) survival after SEQ ID NO: 1 also known as NRP2945 was applied 16× times (twice daily) to a first group of rats starting at 30 min after optic nerve ligation/reperfusion as an eye drop to the cornea of each restrained rat compared to a group of rats rescued by prophylactically applied Brimonidine.

Further aspects of the invention will become apparent with reference to the accompanying Figures and Examples described below:

### EXAMPLE 1

### NRP2945 Efficacy in a Rat Model of Optic Nerve Ligation

### Animals

Male Long-Evans rats (aged P50) were housed for up to 7 days before the start of experimentation and were monitored for signs of ill health. Animals displaying ocular abnormalities were excluded from the study. Every rat was monitored for body weight daily.

### Grouping of Animals for Optic Nerve Ligation Study

Animals were assessed by measuring a baseline electro-retinogram (ERG) at day 0 just before injury in order to normalize all rats in respect of their b-wave amplitude value and group them into three groups as detailed below and as shown in Table 1:

Group 1 received 5 ng of NRP2945 reconstituted in saline twice daily as an eye drop (starting with 30 min after surgery-reperfusion). Only one eye per animal received the injury and drug treatment, while the non-injured eye served as control.

Group 2 received one dose of physiological saline (intra-vitreal route at 30 min after surgery-reperfusion);

Group 3 received the adrenergic α-type 2 agonist brimonidine in prophylactic fashion at a concentration of 1 mg/kg (IP-route at 1 hr before injury).

TABLE 1

| Group | | | Route of admin. | Time of | Number of |
|---|---|---|---|---|---|
| No. | Treatment | Dose | (volume) | admin. | animals |
| 1 | NRP2945 | 5 ng/eye | Right eye topical | Day 0 to Day | 14 |
| | | | instillation (12.5 μl) | 7: twice daily | |
| 2 | Vehicle (Saline) | — | Right eye intravitreal | Day 0 (just | 16 |
| | | | route (5 μl) | after | |
| | | | | ischemia) | |
| 3 | Brimonidine | 1 mg/kg | Intraperitoneal | Day 0: 1 h | 15 |
| | (0.2% w/v) | | 0.5 ml/kg | before | |
| | | | | ischemia | |

### ERG Evaluation and Measurements

ERG measurements were recorded before ischemia (baseline) and 7 days after reperfusion on both eyes in dark-adapted animals. The latency times (for a- and b-wave) and the a-wave and b-wave amplitudes (μV) was measured for each ERG; the a-wave and b-wave amplitudes was expressed as a percentage of the baseline value obtained before ischemia. 15 min before measurement 10 μl Mydriaticum® (0.5% tropicamide) was instilled for pupillary dilatation.

### ERG Parameters:

Color: white maximum.
Maximum intensity: 2.6 cd·s/m2 (0 dB); Duration 0.24 ms; 1 flash

### Filter: 50 Hz.

Impedence Threshold: 90 kΩ.

### Method of Optic Nerve Ligation

Animals were anesthetized by an intramuscular injection of a mix of 2 mg/kg xylazine and 2 mg/kg ketamine. For the vascular ligation model right eyes underwent a temporal orbitectomy combined with periorbital stripping. The globe remained in the orbit and was completely isolated on a pedicle consisting of the optic nerve, ophthalmociliary arteries and the venous outflow. A ligation placed around the pedicle initiated the global ocular ischemia when the ligation was tightened. Ischemia was maintained for 45 minutes. The reperfusion period was initiated by the release of the ligation.

### Study Termination and RGC Evaluation

At the end of the study, the animals were euthanized by intraperitoneal injection of overdosed pentobarbital. After euthanasia at day 8 after optic nerve ligation injury, the retinae of both eyes of N=6 animals per cohort were fixed in formalin 4% (1 h at room temperature), dissected and flat-mounted. The flat-mounted preparation was incubated with an Alexa 594 conjugated anti-BRN3A (Brain-specific homeobox/POU domain protein 3A, Chemicon, cat #mAb1585) to visualize the Retinal Ganglion Cells (RGC). Fluorescence was assessed by an Apotome microscope at magnification×20 (Zeiss) within twelve randomly selected respective microscopic fields per subject. The number of surviving RGC was determined with Axio Vision 4.2 software in the respective retinae areas.

### Results

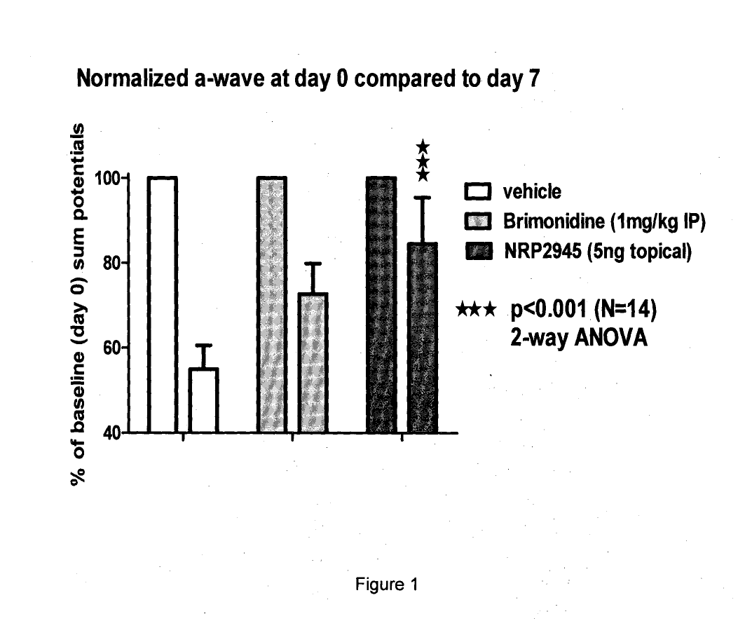

As shown in FIG. 1, wherein SEQ ID NO: 1 also known as NRP2945, was administered as an eye drop (twice daily) starting at 30 min after optic nerve ligation/reperfusion injury, a recovery of 84.5% of the initial a-wave on day 7 after injury was measured. In comparison, the prophylactically administered Brimonidine led to a 72.6% recovery, while the vehicle conditions only led to a 57.0% recovery of the initial a-wave amplitude.

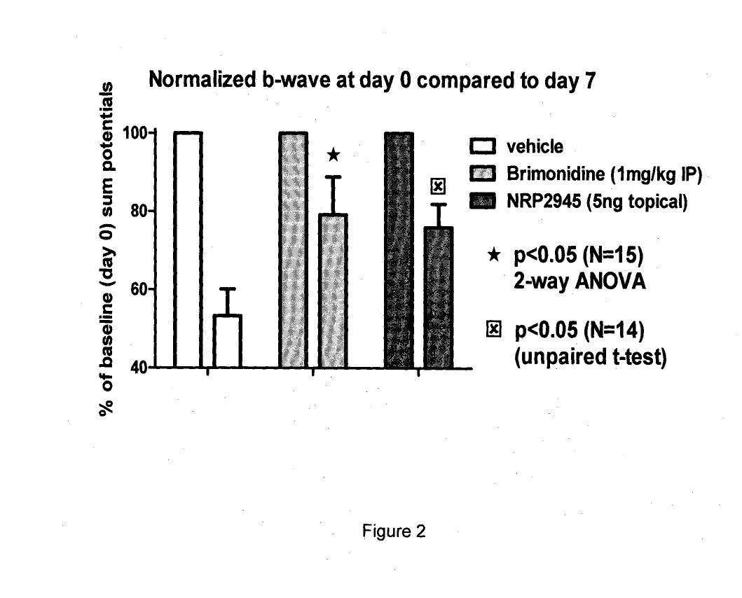

As shown in FIG. 2, SEQ ID NO: 1 also known as NRP2945 was administered as an eye drop (twice daily) starting at 30 min after optic nerve ligation/reperfusion injury, which led to a recovery of 75.8% of the initial b-wave on day 7 after injury. In comparison, the prophylactically administered Brimonidine led to 79.1% recovery, while the vehicle conditions only led to a 58.4% recovery of the initial b-wave amplitude.

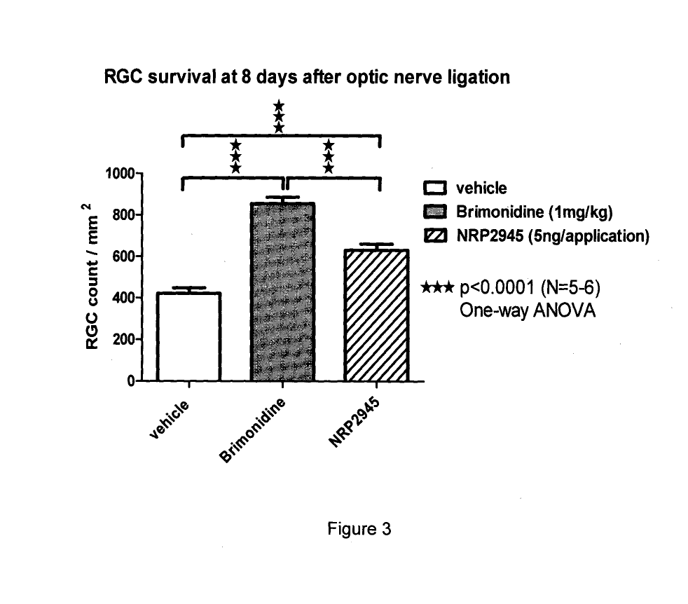

As shown in FIG. 3, SEQ ID NO: 1 also known as NRP2945 was applied 16× times (twice daily) starting at 30 min after optic nerve ligation/reperfusion as an eyedrop to the cornea of a restrained rat. Animals were sacrificed at day 8 and retinae were analysed for BrN3A (Brain-specific homeobox/POU domain protein 3A) protein expression patterns that are specific for retinal ganglion cells (RGCs). 12 fields per retina were evaluated.

The mean RGC density in retina of non-ischemic eyes in this assay was 2158 RGCs/mm2 (n=18). RGC density decreased to 421.0±25.9 RGCs/mm2 at 8 days after ischemia (19.6% compared to non-ischemic contralateral eyes) in saline treated group). NRP2945 cohorts showed 629.8±30.3 cells/mm2 (p<0.001, n=6) at 8 days after injury.

NRP2945 rescued 29.2% of total RGCs compared to 39.6% (855.5±30.7 RGCs/mm2 with p<0.001, n=5) of cells rescued by prophylactically applied Brimonidine. All differences between the respective cohorts are highly statistically significant.

Although the invention has been described with reference to an embodiment or example it should be appreciated that variations and modifications may be made to this embodiment or example without departing from the scope of the invention.

Where known equivalents exist to specific features, such equivalents are incorporated as if specifically referred to in this specification.

In particular, it is anticipated that functionally similar peptide sequences may be obtained by substitution of one or more amino acids of the biosequence with a functionally similar amino acid. It is suggested that the functionality of similar peptide sequences may be confirmed without undue additional experimentation by use of the method disclosed in this specification.

### REFERENCES

Soto, I., Oglesby, E., Buckingham, B. P., Son, J. L., Roberson, E. D. O., Steele, M. R., Inman, D. M., Vetter, M. L., Homer, P. J. and Marsh-Armstrong, N. (2008). Retinal Ganglion Cells down-regulate gene expression and lose their axons within the optic nerve head in a mouse glaucoma model. J Neurosci 28: 548-561.
Prokosch, V., Panagis, L., Volk, G. F., Dermon, C. and Thanos, S. (2010). α-2-adrenergic receptors and their core involvement in the process of axonal growth in retinal explants. Invest. Ophthalmology 51: 6688-6699.
Weber, B., Steinfath, M., Scholz, J. and Bein, B. (2007). Neuroprotective effects of α-2-adrenergic receptor agonists. Drug News Perspect 20: 149-154.
Yoles, E., Wheeler, L. A. and Schwartz, M. (1999). α-2-adrenoreceptor agonists are neuroprotective in a rat model of optic nerve degeneration. Invest Ophthalmol Vis Sci 40: 65-73.
Levkovitch-Verbin, H., Harris-Cerruti, C., Groner, Y., Wheeler, L. A., Schwartz, M. and Yoles, E. (2000). RGC death in mice after optic nerve crush injury: oxidative stress and neuroprotection. Invest Ophthalmol Vis Sci 41: 4169-4174.
Loenngren, U., Naepaenkangas, U., Lafuente, M., Mayor, S., Lindqvist, N., Vidal-Sanz, M. and Halibook, F. (2006). The growth factor response in ischemic rat retina and superior colliculus after brimonidine pre-treatment. Brain Res Bulletin 71: 208-218.

1-44. (canceled)

45. A method of treating or preventing optic nerve damage in a subject comprising administering an effective amount of a peptide comprising the sequence
GlyArgArgAlaAlaProGlyArgAibGlyGly (SEQ ID NO:1) to an eye of a subject in need thereof.

46. The method as claimed in claim 45 wherein the peptide consists of the 11 amino acid residue sequence:
GlyArgArgAlaAlaProGlyArgAibGlyGly
and one or more ophthalmologically acceptable derivatives thereof.

47. The method as claimed in claim 46 wherein one such ophthalmically acceptable derivative includes the sequence wherein the C-terminus of the peptide is amidated to give:
(SEQ ID NO: 2)
GlyArgArgAlaAlaProGlyArgAibGlyGly-NH₂.

48. The method as claimed in claim 45 wherein the administration step to the subject is by way of one or more topically applied eye drops.

49. The method as claimed in claim 45 wherein the administration step to the subject's eye is by way of administration of a cream or an ointment.

50. The method as claimed in claim 45 wherein the administration step to the subject's eye is by way of a liquid formulation applied to the conjunctival sac of the eye of the subject.

51. The method as claimed in claim 45 wherein the administration step to the subject's eye is by way of an intravitreal injection.

52. The method of claim 45 wherein an effective amount of a peptide comprising the sequence:
(SEQ ID NO: 1)
GlyArgArgAlaAlaProGlyArgAibGlyGly
or
(SEQ ID NO: 2)
GlyArgArgAlaAlaProGlyArgAibGlyGly-NH₂
is administered to an eye of the subject in need thereof on an at least once a day basis.

53. The method of claim 52 wherein the peptide is administered to an eye of the subject in need thereof on an at least twice a day basis.

54. The method of claim 45 including the step of administering an effective amount of a peptide comprising the sequence:
(SEQ ID NO: 1)
GlyArgArgAlaAlaProGlyArgAibGlyGly
to an eye of the subject in need thereof on an at least once a day basis.

55. The method of claim 54 wherein the peptide is administered to an eye of the subject in need thereof on an at least twice a day basis.

56. The method of claim 45 wherein the subject is selected from the group consisting of: humans and companion animals.

57. A method of treating or preventing an ophthalmic reperfusion injury in a subject comprising administering an effective amount of a peptide comprising the sequence:
GlyArgArgAlaAlaProGlyArgAibGlyGly (SEQ ID NO:1) to an eye of a subject in need thereof.

58. The method as claimed in claim 57 wherein the peptide consists of the 11 amino acid residue sequence:
GlyArgArgAlaAlaProGlyArgAibGlyGly
and one or more ophthalmologically acceptable derivatives thereof.

59. The method as claimed in claim 58 wherein the ophthalmically acceptable derivative includes the sequence wherein the C-terminus of the peptide is amidated to give:
(SEQ ID NO: 2)
GlyArgArgAlaAlaProGlyArgAibGlyGly-NH₂.

60. The method as claimed in claim 57 wherein the administration step to the subject is by way of one or more topically applied eye drops.

61. The method as claimed in claim 57 wherein the administration step to the subject's eye is by way of administration of a cream or an ointment.

62. The method as claimed in claim 57 wherein the administration step to the subject's eye is by way of a liquid formulation applied to the conjunctival sac of the eye of the subject.

63. The method as claimed in claim 57 wherein the administration step to the subject's eye is by way of an intravitreal injection.

64. The method as claimed in claim 57 including the step of administering an effective amount of a peptide comprising the sequence:
(SEQ ID NO: 1)
GlyArgArgAlaAlaProGlyArgAibGlyGly
or
(SEQ ID NO: 2)
GlyArgArgAlaAlaProGlyArgAibGlyGly-NH₂
to an eye of the subject in need thereof on an at least once a day basis.

65. The method of claim 64 wherein the peptide is administered to an eye of the subject in need thereof on a at least twice a day basis.

66. The method as claimed in claim 57 including the step of administering an effective amount of a peptide comprising the sequence:
(SEQ ID NO: 1)
GlyArgArgAlaAlaProGlyArgAibGlyGly
to an eye of the subject in need thereof on a once a day basis.

67. The method of claim 66 wherein the peptide is administered to an eye of the subject in need thereof on a at least twice a day basis.

68. The method as claimed in claim 57 wherein the subject is selected from the group consisting of: humans and companion animals.

69. A method of treating or preventing ophthalmic ischemia in a subject comprising administering an effective amount of a peptide comprising the sequence:
GlyArgArgAlaAlaProGlyArgAibGlyGly (SEQ ID NO:1) to an eye of a subject in need thereof.

70. The method as claimed in claim 69 wherein the peptide consists of the 11 amino acid residue sequence:
GlyArgArgAlaAlaProGlyArgAibGlyGly
and one or more ophthalmologically acceptable derivatives thereof.

71. The method as claimed in claim 70 wherein the ophthalmically acceptable derivative includes the sequence wherein the C-terminus of the peptide is amidated to give:
(SEQ ID NO: 2)
GlyArgArgAlaAlaProGlyArgAibGlyGly-NH₂.

72. The method as claimed in claim 69 wherein the administration step to the subject is by way of one or more topically applied eye drops.

73. The method as claimed in claim 69 wherein the administration step to the subject's eye is by way of administration of a cream or an ointment.

74. The method as claimed in claim 69 wherein the administration step to the subject's eye is by way of a liquid formulation applied to the conjunctival sac of the eye of the subject.

75. The method as claimed in claim 69 wherein the administration step to the subject's eye is by way of an intravitreal injection.

76. The method as claimed in claim 69 including the step of administering an effective amount of a peptide comprising the sequence:
(SEQ ID NO: 1)
GlyArgArgAlaAlaProGlyArgAibGlyGly
or
(SEQ ID NO: 2)
GlyArgArgAlaAlaProGlyArgAibGlyGly-NH₂
to an eye of the subject in need thereof on an at least once a day basis.

77. The method of claim 76 wherein the peptide is administered to an eye of the subject in need thereof on an at least twice a day basis.

78. The method as claimed in claim 69 including the step of administering an effective amount of a peptide comprising the sequence:
(SEQ ID NO: 1)
GlyArgArgAlaAlaProGlyArgAibGlyGly
to an eye of the subject in need thereof on a once a day basis.

79. The method of claim 78 wherein the peptide is administered to an eye of the subject in need thereof on an at least twice a day basis.

80. The method as claimed in claim 69 wherein the subject is selected from the group consisting of: humans and companion animals.

81. The use in the manufacture of a medicament of an effective amount of at least one of the peptides selected from:
(SEQ ID NO: 1)
GlyArgArgAlaAlaProGlyArgAibGlyGly
or
(SEQ ID NO: 2)
GlyArgArgAlaAlaProGlyArgAibGlyGly-NH₂
for treating or preventing
(i) optic nerve damage;
(ii) an ophthalmic reperfusion injury; or
(iii) ophthalmic ischemia; in an eye of a subject in need thereof.

82. The use as claimed in claim 81 wherein the medicament is adapted for topical administration as an eye drop to an eye of the subject in need thereof.

83. The use as claimed in claim 81 wherein the medicament is adapted for topical ophthalmic administration in the form of a cream or an ointment.

84. The use as claimed in claim 81 wherein the medicament is adapted as a liquid formulation for application to the conjunctival sac of the eye of the subject.

85. The use as claimed in claim 81 wherein the medicament is adapted as a liquid formulation for application to the subject's eye by way of an intravitreal injection.

86. The use as claimed in claim 81 wherein the medicament is adapted for once a day administration.

87. The use as claimed in claim 81 wherein the medicament is adapted for at least twice daily administration.

88. The use as claimed in claim 81 wherein the subject is selected from the group consisting of: humans and companion animals.
